(19) United States
(12) Patent Application Publication
(10) Pub. No.: US 2022/0067050 A1
(43) Pub. Date: Mar. 3, 2022

(54) HUNTING TAG VALIDATION SYSTEM THAT INCORPORATES ANALYSIS OF AN ANIMAL SAMPLE

(71) Applicant: Mark AYERS, Los Osos, CA (US)

(72) Inventor: Mark AYERS, Los Osos, CA (US)

(21) Appl. No.: 17/009,706

(22) Filed: Sep. 1, 2020

(51) Int. Cl. G06F 16/2457; G01N 33/483; G01N 33/49

(52) U.S. Cl. CPC G06F 16/24575; G01N 33/49; G01N 33/4833

(57) ABSTRACT

A system that verifies that a hunter has complied with the conditions of a hunting tag by requiring the hunter to submit a sample (such as a blood sample) of the killed animal with a harvest report; the sample is analyzed to develop a profile of the animal killed, which is then compared to the authorized kills associated with the hunting tag. Sample analysis may determine for example the species, subspecies, or other category of the animal, its sex, its approximate age, the development of body parts such as antlers, and when the animal was killed. Comparison of this data to hunting tag restrictions may be fully or partially automated, enabling efficient flagging of potential violations. Sample analysis may generate a DNA fingerprint that may be used later to cross-check against other animal samples, for example to check trophies or meat to see if they come from verified legal hunts.

### BACKGROUND OF THE INVENTION

### Field of the Invention

One or more embodiments of the invention are related to the fields of hunting equipment and data processing. More particularly, but not by way of limitation, one or more embodiments of the invention enable a hunting tag validation system that incorporates analysis of an animal sample.

### Description of the Related Art

In many countries and regions, hunters must obtain specific permits before hunting many animals. These permits, often known as hunting tags, typically authorize a hunter to hunt a specific quantity of a specific type of animal during a particular time period. Often hunters are required to report on the results of their hunts.

Verification that hunters comply with the conditions of their hunting tags is currently limited. Game wardens may for example perform ad hoc inspections in the field to determine whether a killed animal was obtained legally. However, there are no existing systematic procedures to check whether hunting tag restrictions are being followed. The hunting system relies largely on the integrity of the hunters themselves. This situation often allows unscrupulous hunters to take unauthorized game without detection. The issuance of hunting tags and required reporting on hunt results provides only partial control, since unscrupulous hunters can report false results. As a simple example, a deer hunting tag may allow hunting only of male deer; a hunter may illegally kill a female deer but report that he or she has legally killed a male deer. Currently the only control to prevent this type of unauthorized hunting is field inspection.

For at least the limitations described above there is a need for a hunting tag validation system that incorporates analysis of an animal sample.

### BRIEF SUMMARY OF THE INVENTION

One or more embodiments described in the specification are related to a hunting tag validation system that incorporates analysis of an animal sample. Embodiments of the invention may collect a sample of an animal killed by a hunter, and may analyze this sample to determine whether the hunter complied with the conditions of his or her hunting tag.

One or more embodiments of the invention may include a hunting tag issued to a hunter, and a harvest report linked to the hunting tag. The harvest report may have a sample collection area into which a hunter or other party may place a sample of an animal killed by the hunter, such as a blood sample for example. The hunting tag may have a hunting tag identifier that is also on the harvest report. The harvest report may be transmitted to a sample analysis service that analyzes the sample in the harvest report and generates a harvest sample analysis report with a profile of the animal killed. The harvest sample analysis report may also contain the hunting tag identifier so that it may be cross-referenced to the hunting tag. The harvest sample analysis report may be transmitted to a computer that is connected to a database of hunting tag information. This computer may retrieve a hunting tag record in the database, for example using the hunting tag identifier as a key. This hunting tag record may describe the authorized kills allowed by the hunting tag. For example, in deer hunting with one animal allowed per tag, embodiments of the invention may be utilized to determine if multiple animals have been harvested in violation of the tag. One or more embodiments thus thwart attempts at “tag reuse”. The computer may compare the authorized kills allowed by the hunting tag to the profile of the killed animal in the harvest sample analysis report to determine whether the kill was authorized by the hunting tag.

The sample collection area of the harvest report may be configured to receive and in one or more embodiments, preserve any type of sample from the killed animal, including but not limited to a blood sample, a flesh sample, a hair sample, and a horn or antler sample. In one or more embodiments, separate areas on the sample collection area may be utilized for each animal that the tag is valid for. Thus, there may be one or more sample collection areas per instance of the device. In other embodiments, the sample collection area is configured to receive and preserve multiple samples from different animals harvested in accordance with the tag quota. In one or more embodiments, a fixative may be utilized in conjunction with the sample collection area to ensure the sample integrity. Embodiments of the sample collection area may be utilized in conjunction with desiccants or other object or modes of preservation.

In one or more embodiments, the profile of the animal in the harvest sample analysis report may include a DNA analysis of the DNA in the sample. This DNA analysis may include for example, without limitation, a category that the animal belongs to and a sex of the animal. The category may be for example, without limitation, a species, a subspecies, a population, a subpopulation, a genus, a family, and a breed. The comparison of the animal profile to the authorized kills in the hunting tag record may determine whether the category of animal indicated by the DNA is included in the category allowed by the hunting tag, and whether the sex of the animal killed indicated by the DNA is allowed by the hunting tag.

In one or more embodiments the animal profile in the harvest sample analysis report may include an age range of the animal, and this range may be compared to an age range allowed by the hunting tag; if the two ranges overlap then the kill is authorized by the tag.

In one or more embodiments the animal profile in the harvest sample analysis report may include a range of development of a body part of the animal, and this range may be compared to a range allowed by the hunting tag; if the two ranges overlap then the kill is authorized by the tag. The body part may be for example antlers, and the range of development may be the number of forks in the antlers.

In one or more embodiments the animal profile in the harvest sample analysis report may be a time range when the animal was killed, and this time range may be compared to a valid time range for the hunting tag; if the two ranges overlap then the kill is authorized by the tag.

In one or more embodiments the harvest report may have reactants that commence a reaction when the sample is added to the sample collection area or is closed after receiving the sample. The harvest sample analysis report may include a time range when the animal was killed, which may be determined by calculating when the reaction commenced. The time range of when the animal was killed may be compared to a valid time range for the hunting tag; if the two ranges overlap then the kill is authorized by the hunting tag.

In one or more embodiments the harvest report may include a seal that covers the sample collection area. The seal may be transparent. It may include a tamper indicator that indicates whether it has been opened or resealed after it was sealed.

One or more embodiments may further include an inspection kit that may be used by an inspector to collect a sample of a carcass that the hunter asserts is associated with a harvest report. A sample may be taken for example, without limitation, from antlers, flesh, blood, or hair of the carcass. The inspection kit may have an inspection sample collection area that protects a sample until it is analyzed by the sample analysis service. The sample analysis service may generate a profile of the carcass based on analysis of the sample, and this profile may be transmitted in an inspection sample analysis report. The profile in the inspection sample analysis report may be compared to the profile of the animal previously obtained in the harvest sample analysis report to determine whether they match, in order to determine whether the carcass is part of the animal killed by the hunter and reported with the harvest report. The profile of the animal in the harvest sample analysis report may include for example a DNA fingerprint, which may be compared to a DNA fingerprint of the carcass sample in the inspection sample analysis report.

### BRIEF DESCRIPTION OF THE DRAWINGS

The above and other aspects, features and advantages of the invention will be more apparent from the following more particular description thereof, presented in conjunction with the following drawings wherein:

FIG. 1 shows a high-level architecture diagram of an embodiment of the invention, illustrating a hunting harvest report that accepts a blood sample, a sample analysis service that analyzes the blood, and a system that checks the analysis against the hunting tag.

FIG. 2 shows an alternative harvest report that accepts a sample of other body parts such as flesh or hair.

FIG. 3 shows an illustrative sample analysis service that sequences DNA to determine the animal's category and that determines the sex of the animal.

FIG. 4 shows an illustrative sample analysis service that analyzes DNA and blood to estimate the animal's age, the stage of antler development (for example for a deer), and the time the animal was killed.

FIG. 5A illustrates steps in adding a blood sample to a sample collection area of a harvest report; it also shows an illustrative chemical clock mechanism integrated into the harvest report to date when the sample was added.

FIG. 5B continues the example of FIG. 5A to illustrate how the chemical clock in the harvest report may be used to determine whether the kill occurred during a time period authorized by the hunting tag.

FIG. 6 shows an illustrative scenario of an inspector using an inspection kit to collect a flesh sample for validation against a hunting tag.

FIG. 7 continues the example of FIG. 6 to show analysis of the inspection sample collected by an inspector, and comparison of this analysis to data from the previously analyzed harvest report.

FIG. 8 shows illustrative computer hardware that may be used in one or more embodiments of the invention to execute process steps and to store, retrieve, and transmit data.

### DETAILED DESCRIPTION OF THE INVENTION

A hunting tag validation system that incorporates analysis of an animal sample will now be described. In the following exemplary description, numerous specific details are set forth in order to provide a more thorough understanding of embodiments of the invention. It will be apparent, however, to an artisan of ordinary skill that the present invention may be practiced without incorporating all aspects of the specific details described herein. In other instances, specific features, quantities, or measurements well known to those of ordinary skill in the art have not been described in detail so as not to obscure the invention. Readers should note that although examples of the invention are set forth herein, the claims, and the full scope of any equivalents, are what define the metes and bounds of the invention.

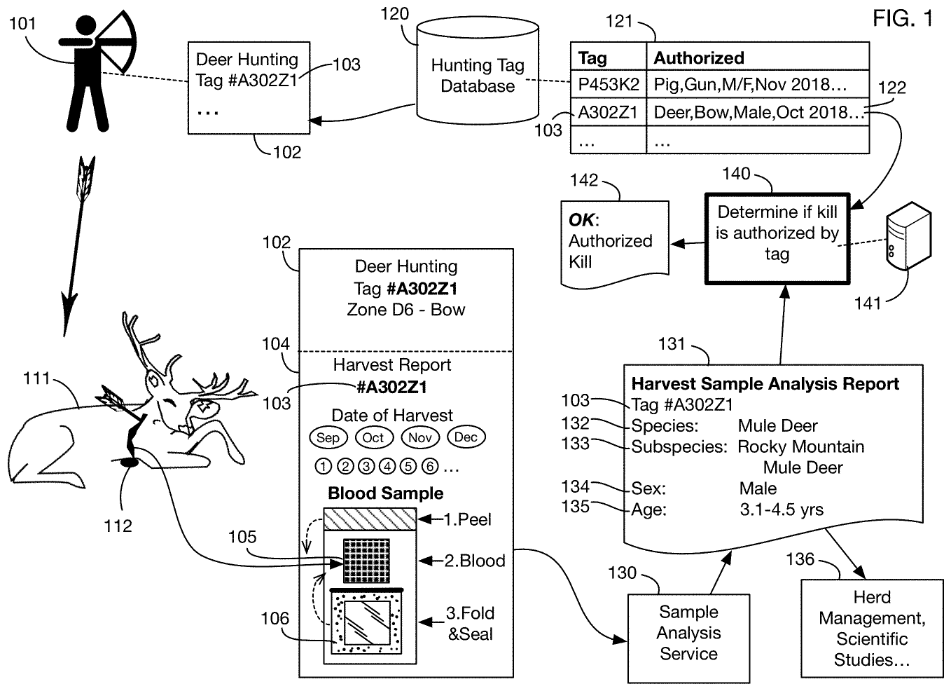

FIG. 1 shows an overview of an embodiment of the invention. A primary function of this embodiment is to determine whether a hunter has complied with the conditions of his or her hunting tag. Hunter 101 obtains a hunting tag 102 that authorizes hunting under certain conditions and restrictions. These conditions may for example be described in one or more databases 120. Database or databases 120 may be data, files, or records in any formats, including but not limited to traditional database formats such as relational databases. For illustration, FIG. 1 shows database 120 as including a table 121 that links hunting tags to the actions authorized by each tag (or prohibited by each tag). In one or more embodiments this information may be spread over multiple tables, files, databases, or data structures. Table 121 has a key that is a hunting tag identifier. This identifier 103 also appears on hunting tag 102. In one or more embodiments a hunting tag identifier may be any information that may be used to correlate a tag or a harvest report with information on the conditions associated with a tag, license, permit, allowance, or authorization. Tag 102 with hunting tag identifier 103 is associated in table 121 with authorizations 122. These authorizations (or equivalently, restrictions) may include limits for example on the days or times when hunting can occur, the types of weapons or ammunition that may be used, the types of animal that can be killed, the sex or age of the animal that can be killed, the stage of development of the animal or any of its body parts, or the region in which hunting can be performed. These categories are illustrative; database 120 and table 121 may associate a hunting tag with any type of information that defines the limits or rights associated with the tag.

A hunting tag may be any right granted to a hunter to hunt, even if it is not referred to as a “tag” by the authority granting the right. For example, in one or more embodiments a hunting tag may be referred to as a license or a permit. In one or more embodiments a hunting tag, license, permit, or right may be assigned to an organization, group, or class instead of to an individual.

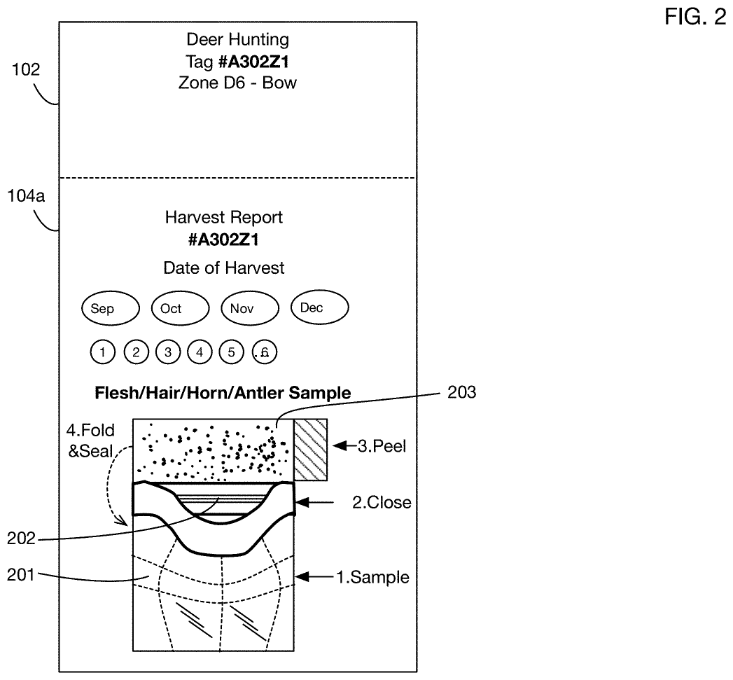

In the illustrative scenario shown in FIG. 1, hunter 101 kills animal 111. Associated with hunting tag 102 is a harvest report 104 that must be submitted by the hunter when the hunter makes a kill. A novel feature of the harvest report 104 is that it includes a sample collection area that the hunter must use to provide a sample of the animal killed. In this example the sample collection area 105 is a pad for collection of a blood sample; FIG. 2 shows a harvest report that may be used to collect other types of samples. The hunter takes blood sample 112 from animal 111 after the kill and places a portion of the blood sample onto area 105. The hunter then attaches a seal 106 over the pad 105 to protect and preserve the blood sample until it is analyzed. The hunter may also fill out or punch information on the harvest report such as the date of the kill. The harvest report includes hunting tag identifier 103 so that it may be cross-referenced to the tag information in database 120. In one or more embodiments the hunting tag 102 may be physically joined to the harvest report 104 when issued; in one or more embodiments the hunting tag and the harvest report may be physically separate, or the hunting tag may be virtual (for example emailed or obtained as an online confirmation), and the harvest report may be linked to the real or virtual hunting tag by the shared hunting tag identifier.

After adding the sample of blood 112 to sample collection area 105 of harvest report 104, hunter 101 (or another person or organization) transmits the harvest report to a sample analysis service or services 130. This sample analysis service may be for example part of or contracted by an agency that manages hunting or wildlife. It may be a single service, or a collection of services that provide various stages or types of analysis. If the hunting tag 102 is physically coupled to the harvest report 104, the hunter may detach the two pieces before transmitting the harvest report, in order to keep the hunting tag with the killed animal 111. In some situations, the hunter may physically transport the animal 111 to an agent such as a game warden, who may then complete the harvest report and transmit it with the sample to the sample analysis service. The sample analysis service 130 analyzes the blood sample (or other sample from the animal 111) and generates a harvest sample analysis report 131. This report 131 may contain a profile of the animal killed with any information that may be determined from the sample provided with the harvest report 104. FIG. 1 shows an illustrative animal profile that may be provided in one or more embodiments, which includes the species 132 and subspecies 133 of the animal, its sex 134, and a range 135 for the animal's age. These profile attributes are illustrative examples; the specific attributes and characteristics that are measured and reported may depend on the requirements associated with the hunting tag and on the capabilities of a particular sample analysis service.

The harvest sample analysis report 131 may then be transmitted to a system 141 that compares the animal profile and data in this report to the authorized kills allowed by the associated hunting tag. This comparison provides for a determination 140 of whether the kill of the animal by the hunter appears to be authorized by the tag. The harvest sample analysis report 131 may for example have a field 103 for the hunting tag identifier or equivalent information, which may be used to obtain the hunting tag record in database 120 associated with this hunting tag identifier. The data 122 in this record that describes the authorized kills allowed by the hunting tag may be correlated with the data in the report 131 to determine if the kill is authorized 142, or if it appears the kill may be illegal based on the hunting tag. As a simple example, if the hunting tag 102 allows only killing of male deer, and if the sample analysis service's analysis indicates that the blood 112 in sample collection area 105 is from a female deer, then the comparison 140 may flag the kill as illegal or as requiring further investigation.

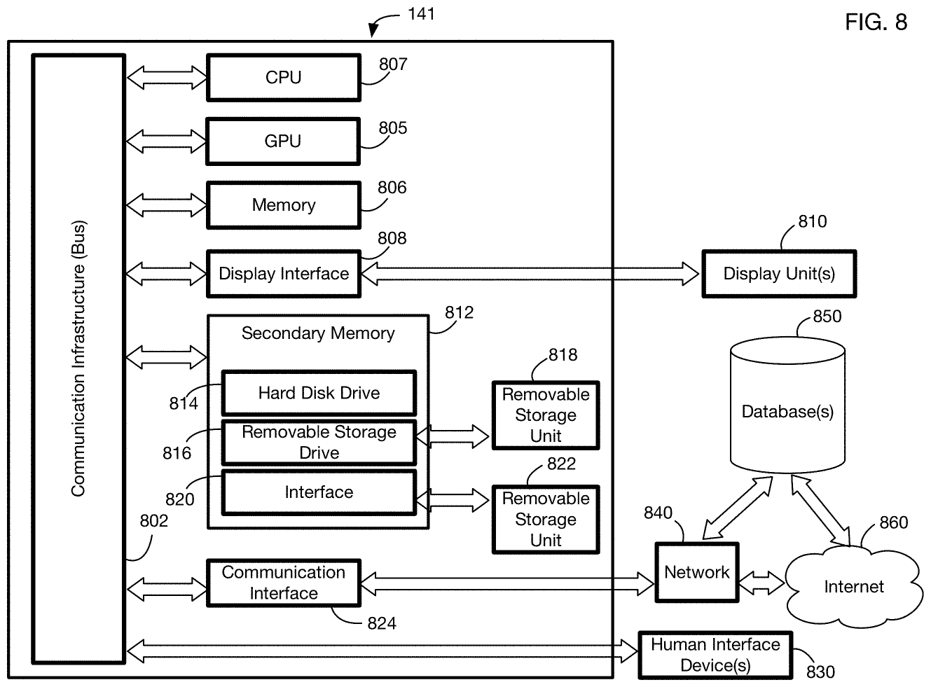

System 141, which executes the assessment 140 of the sample analysis report 131 compared to the data in database 120, may be any computer system or systems. An illustrative computer system that may be used in one or more embodiments is described below with respect to FIG. 8. In one or more embodiments, system 141 may be a collection of computers or a network of computers. In one or more embodiments the harvest sample analysis report 131 may be transmitted electronically to system 141, and the check 140 may be fully automated. In one or more embodiments some data may need to be transferred manually to support the check 140 of the sample analysis results against the hunting tag record 122. In one or more embodiments different systems may perform different portions of the checks 140, for example for different requirements associated with the hunting tag data 122.

The harvest sample analysis report may also be transmitted to other systems or organizations 136. For example, wildlife departments or related agencies may want to monitor the health or status of herds or game populations, and may use harvest sample analysis reports as a valuable source of data. In one or more embodiments the sample analysis service 130 may perform additional analyses for these organizations that are specifically for research or monitoring as opposed to for verification that the kill of the animal is authorized by the hunting tag.

Harvest report 104 of FIG. 1 is configured to accept a blood sample in sample collection area 105. One or more embodiments may have harvest reports that accept other kinds of samples from an animal killed by a hunter.

In one or more embodiments, sample collection area 105 area or areas may be affixed to a preprinted hunting tag 102 or applied or printed on a designated area of the hunting 102. Any other method of coupling sample collection area 105 to hunting tag 102 may be utilized.

FIG. 2 shows an illustrative embodiment of a harvest report 104a with a sample collection area 201 that is a pouch into which a sample of flesh, hair, bone, antler, or other body tissue may be placed. The pouch 201 may contain materials or solutions that preserve the sample until it is analyzed. After inserting the sample into the pouch 201, the hunter may close it using zippers 202 along the edges of the pouch, and then peel a protective backing off of a sealing strip 203 that is folded down over the top of the pouch to seal it. The sealing strip 203 may for example have an adhesive that secures it to the pouch. The seal 203 may be designed to be tamper-evident, for example by sealing with a sufficient force that a user cannot open the seal without leaving a trace that it has been opened. The pouch 201 may be transparent so that the hunter, a warden, or an inspector can observe the contents easily. The pouch 201 and seal 203 may be sufficiently strong that they will protect the sample contents for transport to the sample analysis service.

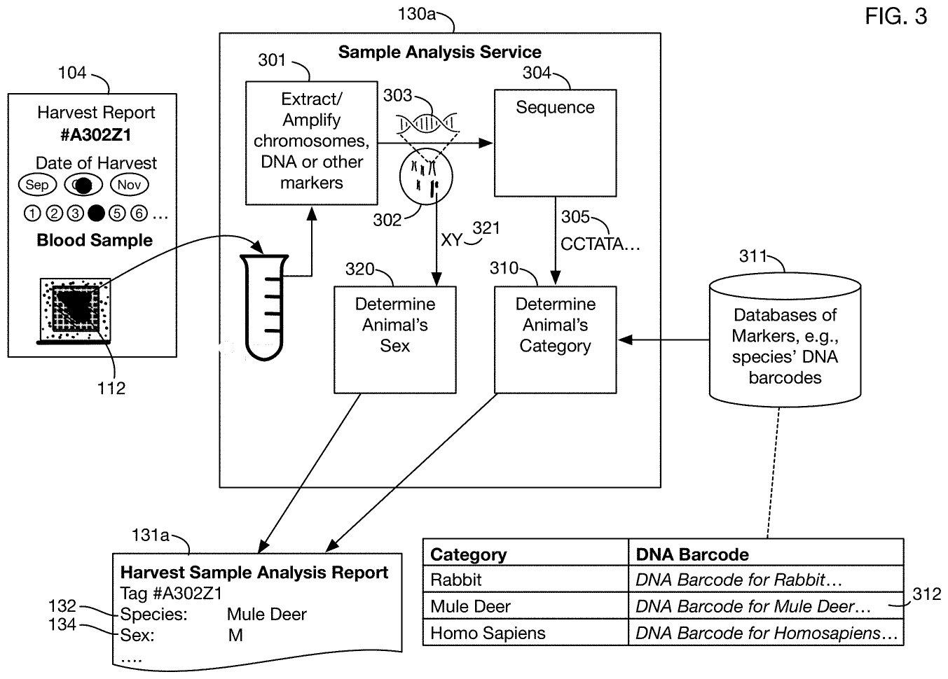

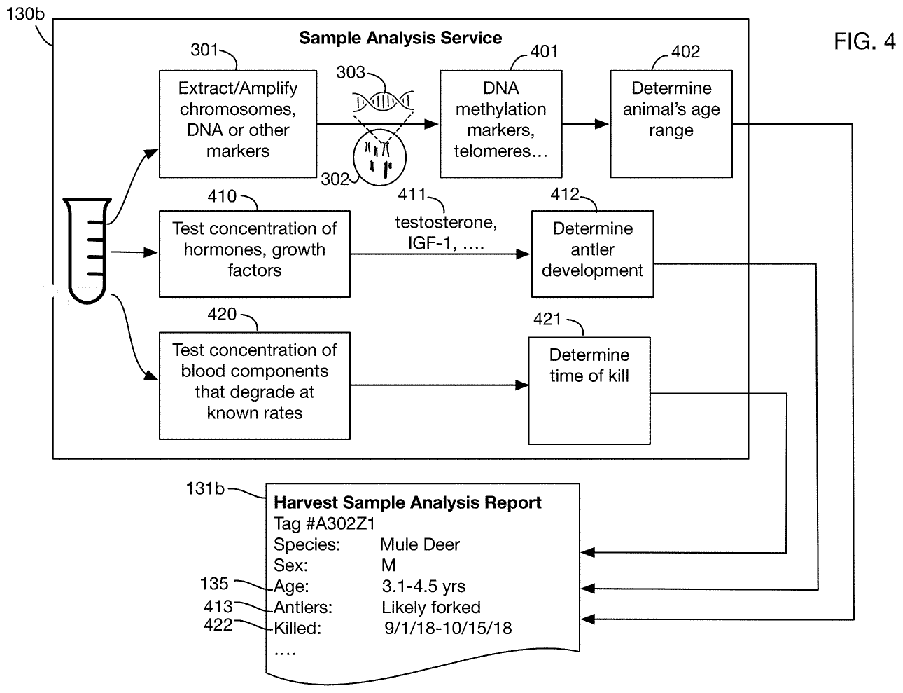

FIGS. 3 and 4 show illustrative analyses that may be performed by a sample analysis service in one or more embodiments. In the embodiment shown in FIG. 3, blood sample 112 from harvest report 104 is analyzed by sample analysis service 130a. The analysis performs extraction step 301 that isolates, extracts, or amplifies chromosomes 302 and DNA 303 in the blood. DNA 303 may be sequenced partially or fully in step 304, resulting in one or more DNA sequences 305. This sequence 305 may be for example compared to a database 311 that contains known markers such as DNA barcodes for species or other animal categories. This comparison may result in a determination 310 of the category or categories the animal belongs to. In this example the DNA sequence 305 matches DNA barcode 312 for species 132, so the harvest sample analysis report 131a identifies this species as the category of the killed animal. In some situations, a sample may be determined to belong to more than one category, either because categories are not mutually exclusive or because the analysis cannot differentiate between certain categories.

Categories in database 311 may correspond to any level of grouping at any level of detail, such as for example any category in any taxonomic hierarchy such as species, genus, or family. Categories may also correspond to levels of detail below a species, such as a subspecies or a population in a particular region, or a breed. The animal category or categories identified in the animal profile of a harvest sample analysis report may also be at any level of aggregation or detail. The reported category or categories may be compared to the data associated with the hunting tag to determine whether the kill was authorized. For example, if a hunting tag authorizes killing animals of one category, then an authorized kill may be an animal determined to be in any category that equals, is a subset of, or overlaps the category associated with the hunting tag.

Sample analysis service 130a also performs a determination 320 of the sex 134 of the animal. This determination may for example analyze the chromosomes 302 to identify sex chromosomes 321. Other blood or genetic markers may be used in one or more embodiments to determine the animal's sex. The reported sex 134 may be compared to the conditions of the hunting tag to determine whether the kill was authorized.

FIG. 4 illustrates additional sample analyses that may be performed in one or more embodiments. A sample analysis service 130b may analyze factors 401 that may contribute to a determination 402 of the animal's age or age range. These factors 401 may include for example DNA methylation, telomeres, or any other biochemical markers that are known to be correlated with an animal's age. The age may be determined within a confidence range 135. This range may be compared to an authorized age range associated with a hunting tag; if these ranges overlap then the kill may be determined to be authorized. In addition to or instead of the animal's age, some hunting tags may require that certain body parts of the animal reach a particular stage of development before it can be legally hunted; for example, a deer tag may authorize kills only of male deer with antlers that are forked, are of a certain size, or that have a certain number of points. In some situations, it may be possible to estimate the stage of development of these body parts with tests 410 that determine concentration of substances 411, such as testosterone or IGF-1, that trigger or reflect growth. For example, an estimate 412 of the stage of development of deer antlers may be made for a deer hunting tag, resulting in data 413 in harvest sample analysis report 131b. In addition, one or more embodiments may perform tests 420 of the sample to determine the concentration of components that are known to degrade or otherwise change after the animal's death; these tests may be used to develop an estimate 421 of the time that the animal was killed 422. This time may be compared to the allowed season associated with a hunting tag to determine whether the kill occurred during the authorized time period; if the range 422 of possible times of the kill does not overlap the authorized range of times for hunting associated with the hunting tag, then the kill may have been illegal.

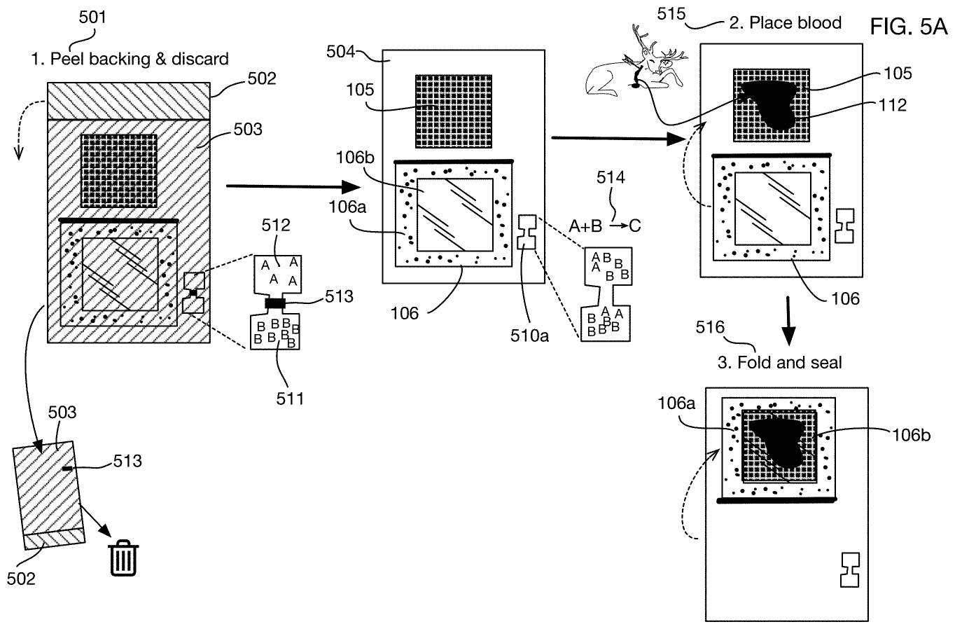

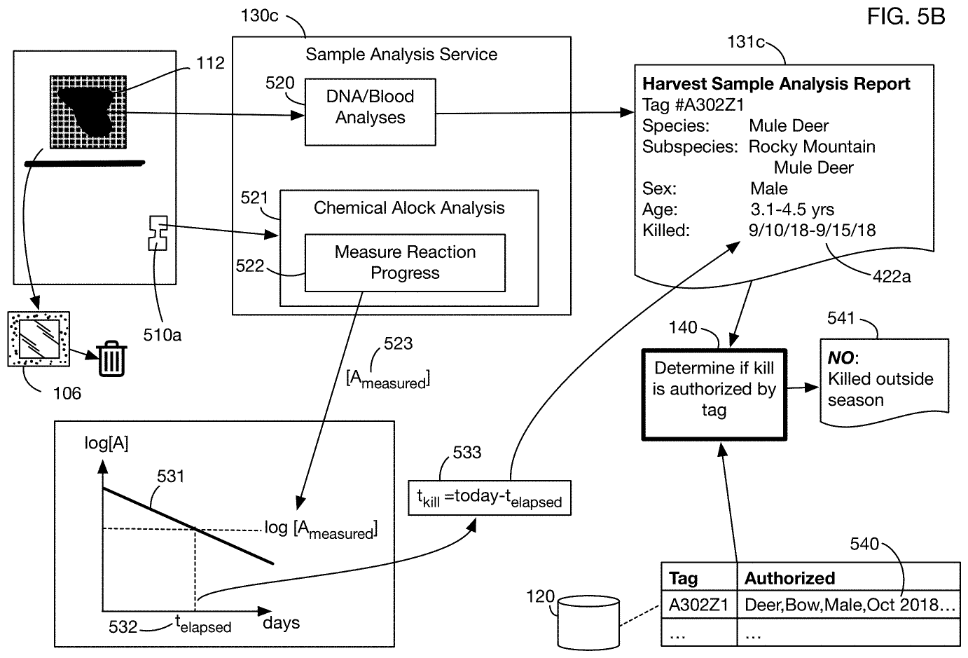

FIG. 5A shows an illustrative sample collection area that may be integrated into a harvest report in one or more embodiments, and it illustrates steps in adding a sample to the sample collection area and securing it for transmission to a sample analysis service. This illustrative sample collection area is configured to receive a blood sample; one or more embodiments may have sample collection areas for other types of samples, as illustrated for example in FIG. 2. Initially the sample collection area is covered by a protective backing. The hunter initially performs step 501 to remove this backing, for example by pulling on tab 502 to peel off a backing 503 that covers the sample collection area. This tab 502 and backing 503 may then be discarded. The exposed sample collection area 504 contains a pad 105 onto which a blood sample may be placed. In one or more embodiments this pad may contain chemicals that preserve the blood until it is analyzed. In one or more embodiments the pad 105 may contain microfluidics channels that receive and route blood into one or more chambers; these chambers may contain various chemicals that preserve the blood or that react with the blood as preparatory steps for subsequent analysis. Sample collection area 504 also contains a seal 106 opposite the pad that is configured to be folded over the pad after the blood sample is added. This seal may have for example a transparent window 106b and an adhesive border 106a that secures the seal around the pad. In step 515 the hunter places blood 112 onto pad 105, and then in step 516 the hunter folds the seal 106 over the pad, securing it with adhesive area 106a.

FIG. 5A illustrates another feature that may be incorporated into a harvest report in one or more embodiments: a chemical clock integrated into the harvest report that may be used to date when the harvest report was completed by the hunter. This chemical clock may be used instead of or in addition to the analyses 420 and 421 described with respect to FIG. 4 to estimate the time of a kill, thereby supporting a determination of whether a kill occurred with a time range allowed by a hunting tag. The chemical clock may be a mechanism that starts a reaction when the hunter completes the harvest report. The illustrative mechanism shown in FIG. 5A is pair of microchambers 511 and 512 integrated into the sample collection area 504. Chamber 511 contains one chemical species denoted “B” and chamber 512 contains another chemical species denoted “A”. The two chambers are connected by a channel, but the channel is blocked by a barrier 513. This barrier is coupled to the backing 503, so that when the hunter removes the backing, the barrier 513 is also removed, connecting the two chambers into a combined chamber 510a. This allows a reaction 514 to begin, which converts the chemicals in the two chambers into a reactant. This reaction proceeds at a known, relatively slow rate, over several days or weeks, so that the time the reaction begins may be estimated as described below with respect to FIG. 5B. One or more embodiments may use different types of mechanisms or chemical clocks instead of or in addition to the one illustrated in FIG. 5A. For example, the reaction 514 may involve more than two reactants, or the chambers 511 and 512 may be located proximal to or under the pad 105 and the barrier between them may be removed when the seal is secured in step 516 rather than when the backing is removed in step 501.

FIG. 5B continues the example of FIG. 5A. At sample analysis service 130c, seal 106 is removed and discarded, exposing the blood sample 112 and the chemical clock area 510a. Blood sample 112 is analyzed in steps 520, for example as described above with respect to FIG. 3 or 4. The contents of chemical clock 510a are analyzed in step 521 with one or more measurements 522 of the progress of the reaction. The measurements 522 may for example measure one or more reactants or products to determine how far the reaction has progressed. For illustration, assuming simple first-order reaction kinetics with reactant “A” as the limiting reactant, and assuming the initial concentration of this reactant is fixed, a measurement 523 of the concentration of this reactant may be sufficient to estimate the progress of the reaction. The reaction rate curve 531 may therefore be used to determine the amount of time 532 that has elapsed since the reaction started. This time 532 may in turn be used to estimate the time 533 that the hunter killed the animal, which may be included as a field 422a in the harvest sample analysis report 131c. In one or more embodiments this estimate 422a may be a range, reflecting imprecision in the measurement 523 or potential variations in the reaction rate, for example due to temperature variation. The range 422a may be compared to the authorized season 540 associated with the hunting tag in database 120, to make determination 541 of whether the kill occurred during the authorized time period.

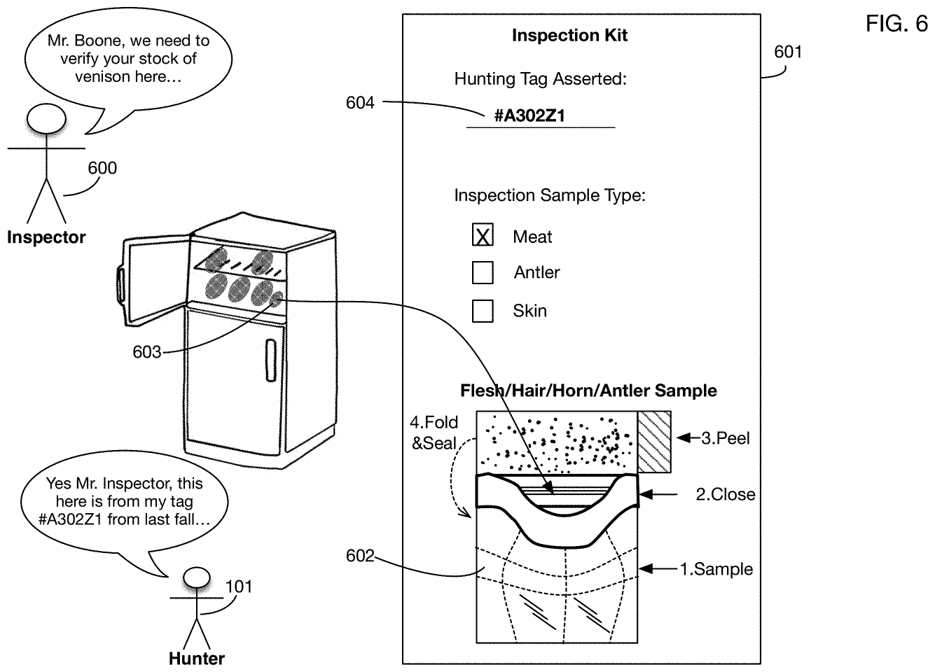

In addition to using an animal sample provided by a hunter to verify that a kill complied with the conditions of a hunting tag, one or more embodiments may provide a capability to perform post-hunt inspection of animal parts to determine whether they are associated with a sample previously provided by a hunter in a harvest report. One or more embodiments may support inspection of any part of an animal carcass, including but not limited to meat, horns, antlers, skin, fur, bones, or any other body parts. For example, a hunter may display trophies such as antlers or mounted heads; these trophies may be inspected by taking a small sample and placing it into the inspection kit. FIG. 6 shows an illustrative scenario with an inspector 600 visiting a hunter 101 to inspect the hunter's stock of meat. Inspector 600 takes a sample 603 from the hunter's stock and places it into an inspection kit 601. The inspection kit may be similar in some respects to the harvest report submitted by a hunter, but it is for use by an inspector instead of by the hunter. The inspector places the sample 603 into a sample collection area 602, which may be similar for example to the sample collection area described with respect to FIG. 2. In this scenario, hunter 101 asserts that the sample is associated with a hunting tag 604 that was issued to the hunter and with the harvest report for that tag that the hunter previously submitted, so the identifier of this hunting tag is included in the inspection kit. The sample collection area 602 is then sealed and the kit 601 is transmitted to a sample analysis service.

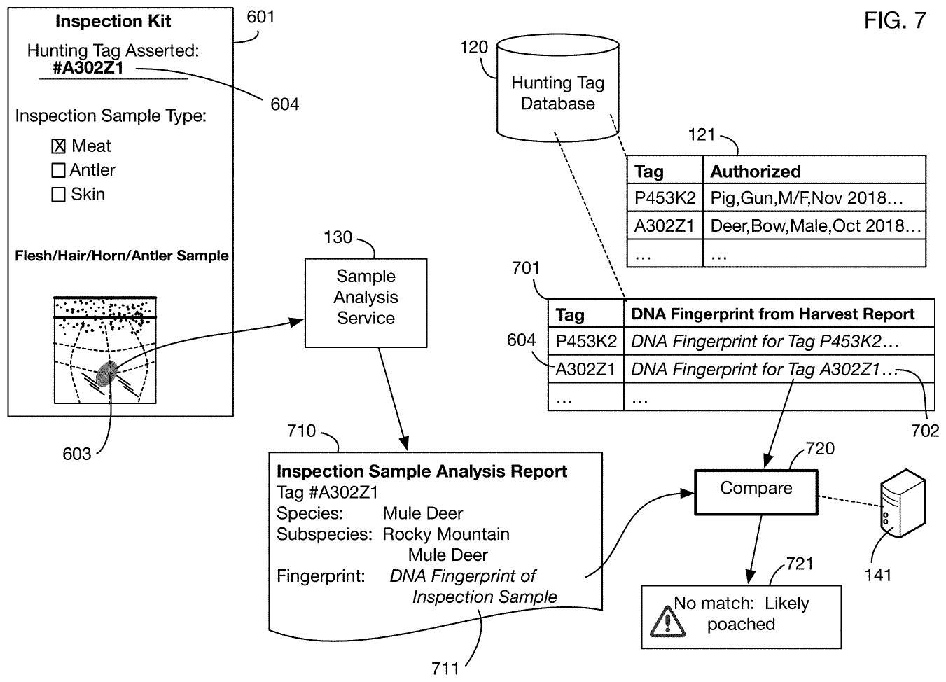

FIG. 7 continues the example of FIG. 6 to illustrate analysis of the inspection kit 601 by sample analysis service 130. Analysis service 130 may perform any type of analysis on the sample from the carcass, to generate any type of profile of the sample. In particular, in one or more embodiments DNA in sample 603 may for example be sequenced to generate a DNA fingerprint 711. This fingerprint 711 may be included in an inspection sample analysis report 710. In one or more embodiments the inspection sample analysis report may contain other information about the sample, such as the species of the animal, sex of the animal, or any other profile data that may be determined from the sample. The inspection sample analysis report 710 is transmitted to system 141 that performs a comparison 720 of this report 710 to data 701 associated with hunting tag identifier 604. This comparison may determine for example if the DNA fingerprint 711 of the inspection sample 603 matches a previously generated DNA fingerprint 702 from a sample provided with a harvest report for that hunting tag. The DNA fingerprint data 702 may be stored for example in the database 120 or in any other database that maintains data from harvest sample analysis reports. In this scenario, the DNA fingerprints 711 and 702 do not match, so the comparison 720 determines that there is a likelihood 721 that the meat in the hunter's possession was obtained illegally.

In one or more embodiments, comparison 720 may compare any animal profile data in inspection sample analysis report 710 with any other data associated with a hunting tag or harvest sample analysis report, including but not limited to DNA fingerprints. Comparison 720 may also perform checks that are independent of any hunting tag; for example, if report 710 indicates that the species of a sample is a species that is not legal to hunt (such as an endangered species), then the meat or other items in the hunter's possession were clearly obtained illegally.

FIG. 8 shows an embodiment of exemplary computer 141 that may be utilized in, by, or as any component in the system. In one or more embodiments, computer 141 may be a network of computers, each of which may have any or all of the components shown in FIG. 8. In one or more embodiments, computer or computers 141 may also be utilized to implement any function in the system, i.e., any step or act or function that executes in any computer or server or engine in the system. Computer 141 may include processor CPU 807 that executes software instructions specifically tailored to the respective functions of embodiments of the invention. The software instructions, otherwise known as computer program instructions, may reside within memory 806. Computer 141 may include processor GPU 805, which may execute graphics instructions or other instructions for highly parallel operations, for example. GPU program instructions may also reside within memory 806. Computer 141 may include display interface 808, which may drive display unit or units 810 of any computer in the system as desired. Some computers 141 may or may not utilize a display. Computer 141 may include communication interface 824, which may include wireless or wired communications hardware protocol chips. In one or more embodiments of the invention communication interface 824 may include telephonic and/or data communications hardware. In one or more embodiments communication interface 824 may include a Wi-Fi™ and/or BLUETOOTH™ wireless communications interface. Any wireless network protocol or type may be utilized in embodiments of the invention. CPU 807, GPU 805, memory 806, display interface 808, communication interface 824, human interface devices 830, secondary memory 812, such as hard disk 814, removable storage 816, secondary memory interface 820 and removable storage units 818 and 822 may communicate with one another over communication infrastructure 802, which is commonly known as a “bus”. Communications interface 824 may communicate over any wired or wireless medium that allows for communication with other wired or wireless devices over network 840. Network 840 may communicate with Internet 860 and/or database or databases 850. Database 850 may be utilized to implement any database described herein.

While the invention herein disclosed has been described by means of specific embodiments and applications thereof, numerous modifications and variations could be made thereto by those skilled in the art without departing from the scope of the invention set forth in the claims.

1. A hunting tag validation system that incorporates analysis of an animal sample, consisting essentially of:
a hunting tag configured to be issued to a hunter of a multiplicity of hunters and comprising a hunting tag identifier that corresponds to a key in a database;
a harvest report linked to the hunting tag and configured to be completed by the hunter of said multiplicity of hunters when the hunter makes a kill of an animal, the harvest report comprising
the hunting tag identifier that corresponds to said key in said database,
a sample collection area configured to
receive a sample of the animal,
protect the sample until it is analyzed,
wherein the harvest report is further configured to be transmitted to a sample analysis service;
a database comprising a hunting tag record for each of said multiplicity of hunters, said hunting tag record corresponding to the hunting tag, the hunting tag record comprising
the hunting tag identifier that corresponds to said key in said database associated with the hunting tag,
authorized kills allowed by the hunting tag; and
a computer coupled with the database and configured to
receive a harvest sample analysis report from the sample analysis service for each of a multiplicity of hunters that respectively make said kill of said animal, the harvest sample analysis report comprising
the hunting tag identifier of the harvest report that corresponds to said key in said database,
a profile of the animal based on analysis of the sample of the animal in the sample collection area of the harvest report;
retrieve the hunting tag record that corresponds to said key in said database for each of a multiplicity of hunters associated with a respective harvest report in the database; and
compare the authorized kills allowed by the hunting tag associated with the hunting tag record for each of said multiplicity of hunters to the profile of the animal of the harvest sample analysis report associated with said each of said multiplicity of hunters to determine whether the kill of the animal is authorized by the hunting tag associated with each of said multiplicity of hunters.

2. The system of claim 1 wherein the sample of the animal comprises a blood sample.

3. The system of claim 1 wherein the sample of the animal comprises a flesh sample.

4. (canceled)

5. The system of claim 1 wherein the sample of the animal comprises a hair or horn or antler sample.

6. The system of claim 1 wherein the profile of the animal comprises a DNA analysis of DNA in the sample of the animal.

7. The system of claim 6 wherein
the DNA analysis comprises
a category of animal indicated by the DNA in the sample of the animal,
a sex of animal indicated by the DNA in the sample of the animal;
the authorized kills allowed by the hunting tag comprises
a category of authorized animals,
a collection of one or more authorized sexes; and
said computer is further configured to
determine whether the category of animal indicated by the DNA in the sample of the animal is included in the category of authorized animals; and
determine whether the sex of animal indicated by the DNA in the sample of the animal is included in the collection of one or more authorized sexes.

8. The system of claim 7 wherein one or both of the category of authorized animals and the category of animal indicated by the DNA in the sample of the animal comprise one or more of a species, a subspecies, a population, a subpopulation, a genus, a family, a breed.

9. The system of claim 1 wherein
the profile of the animal comprises
an age range of the animal;
the authorized kills allowed by the hunting tag comprises
an authorized age range; and,
the determine whether the kill of the animal is authorized by the hunting tag comprises
determine whether the age range of the animal overlaps the authorized age range.

10. The system of claim 1, wherein
the profile of the animal comprises
a range of development of a body part of the animal;
the authorized kills allowed by the hunting tag comprises
an authorized range of development of the body part; and,
the determine whether the kill of the animal is authorized by the hunting tag comprises
determine whether the range of development of the body part of the animal overlaps the authorized range of development of the body part.

11. The system of claim 10, wherein
the body part is antlers;
the development of the body part comprises a number of forks in the antlers; and,
the authorized range of development of the body part comprises a minimum number of forks in the antlers.

12. The system of claim 1 wherein
the profile of the animal based on the analysis of the sample of the animal comprises a time range of when the animal was killed;
the authorized kills allowed by the hunting tag comprises a valid time range for the hunting tag;
the determine whether the kill of the animal is authorized by the hunting tag comprises
determine whether the time range of when the animal was killed overlaps the valid time range for the hunting tag.

13. The system of claim 1 wherein
the harvest report further comprises one or more reactants configured to commence a reaction when the sample collection area receives the sample or is closed after receiving the sample;
the harvest sample analysis report further comprises a time range of when the reaction commenced;
the authorized kills allowed by the hunting tag comprises a valid time range for the hunting tag;
the determine whether the kill of the animal is authorized by the hunting tag comprises
determine whether the time range of when the reaction commenced overlaps the valid time range for the hunting tag.

14. The system of claim 1 wherein the harvest report further comprises
a seal configured to cover the sample collection area.

15. The system of claim 14 wherein the seal is transparent.

16. The system of claim 14 wherein the seal comprises a tamper indicator that indicates that the seal has been opened or resealed after it has been sealed.

17. The system of claim 1 further consisting essentially of:
an inspection kit configured to be completed by an inspector and comprising
an inspection sample collection area configured to
receive an inspection sample of a carcass, associated with the carcass that is part of the animal associated with the harvest report,
protect the inspection sample until it is analyzed;
wherein the inspection kit is further configured to be transmitted to the sample analysis service;
wherein said computer is further configured to
receive an inspection sample analysis report from the sample analysis service, the inspection sample analysis report comprising
a profile of the carcass based on the analysis of the inspection sample of the carcass in the inspection sample collection area of the inspection kit;
compare the profile of the animal to the profile of the carcass to determine whether the carcass is part of the animal killed by the hunter.

18. The system of claim 17 wherein
the profile of the animal comprises a DNA fingerprint of the sample of the animal;
the profile of the carcass comprises a DNA fingerprint of the sample of the carcass;
the computer is further configured to
determine whether the DNA fingerprint of the sample of the animal matches the DNA fingerprint of the sample of the carcass.

19. The system of claim 17 wherein the sample of the carcass is all or a portion of one or more antlers of the carcass.

20. The system of claim 17 wherein the sample of the carcass is flesh.

21. The system of claim 17 wherein the sample of the carcass is blood.

22. The system of claim 17 wherein the sample of the carcass is hair.

23. A hunting tag validation system that incorporates analysis of an animal sample, consisting essentially of:
a hunting tag configured to be issued to a hunter of a multiplicity of hunters and comprising a hunting tag identifier that corresponds to a key in a database;
a harvest report linked to the hunting tag and configured to be completed by the hunter of said multiplicity of hunters when the hunter makes a kill of an animal, the harvest report comprising
the hunting tag identifier that corresponds to said key in said database,
a sample collection area configured to
receive a sample of the animal wherein the sample is blood,
protect the sample until it is analyzed,
wherein the harvest report is further configured to be transmitted to a sample analysis service;
a database comprising a hunting tag record for each of said multiplicity of hunters, said hunting tag record corresponding to the hunting tag, the hunting tag record comprising
the hunting tag identifier that corresponds to said key in said database associated with the hunting tag,
authorized kills allowed by the hunting tag; and
a computer coupled with the database and configured to
receive a harvest sample analysis report from the sample analysis service for each of a multiplicity of hunters that respectively make said kill of said animal, the harvest sample analysis report comprising
the hunting tag identifier of the harvest report that corresponds to said key in said database,
a profile of the animal based on analysis of the sample of the animal in the sample collection area of the harvest report;
retrieve the hunting tag record that corresponds to said key in said database for each of a multiplicity of hunters associated with a respective harvest report in the database; and
compare the authorized kills allowed by the hunting tag associated with the hunting tag record for each of said multiplicity of hunters to the profile of the animal of the harvest sample analysis report associated with said each of said multiplicity of hunters to determine whether the kill of the animal is authorized by the hunting tag associated with each of said multiplicity of hunters;
an inspection kit configured to be completed by an inspector and comprising
an inspection sample collection area configured to
receive an inspection sample of a carcass, associated with the carcass that is part of the animal associated with the harvest report protect the inspection sample until it is analyzed;
wherein the inspection kit is further configured to be transmitted to the sample analysis service;
wherein said computer is further configured to
receive an inspection sample analysis report from the sample analysis service, the inspection sample analysis report comprising
a profile of the carcass based on the analysis of the inspection sample of the carcass in the inspection sample collection area of the inspection kit;
compare the profile of the animal to the profile of the carcass to determine whether the carcass is part of the animal killed by the hunter.
